(19) United States
(12) Patent Application Publication
(10) Pub. No.: US 2020/0230303 A1
(43) Pub. Date: Jul. 23, 2020

(54) PRP PRODUCTION KIT WITH THE ABILITY TO RE-INJECT THE REMAINING ELEMENTS AFTER SEPARATING PRP FROM THE BLOOD

(71) Applicant: Ahmad GHANBARI, Tehran (IR)

(72) Inventor: Ahmad GHANBARI, Tehran (IR)

(21) Appl. No.: 16/632,010

(22) PCT Filed: Jul. 22, 2017
(86) PCT No.: PCT/IB2017/054453

§ 371 Date: Jan. 17, 2020

(51) Int. Cl. A61M 1/02; A61M 1/36; A61M 1/38; A61M 39/10

(52) U.S. Cl. CPC A61M 1/0209; A61M 1/3693; A61M 2202/0427; A61M 1/3672; A61M 39/105; A61M 1/38

(57) ABSTRACT

In the presented invention, by using two connected polymer bags and the connection to the drawing blood, the first separation in the first bag and transmission of the blood components; such as RPP into the second bag and re-injecting the remaining blood to the considered person are possible.

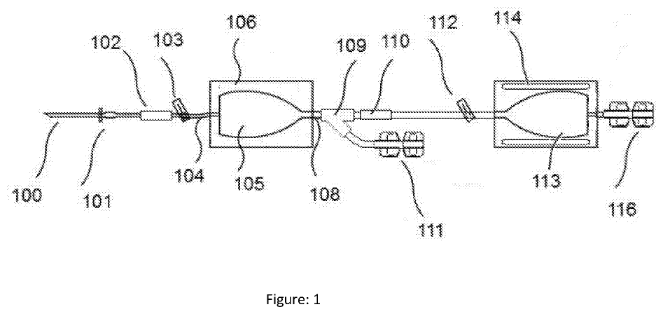

### TECHNICAL FIELD

The presented invention is related to the tool and method for extracting blood from the living creature's body and extracting the platelet element for injecting to the drawing blood subject.

### BACKGROUND OF THE INVENTION

The past researches on the effect on platelet aggregation on the tissues of living organisms and in particular humans are illustrated that increasing the platelet levels caused to stimulate the living cell to rebuild itself and to form of a tissue that fits the original texture. Given this importance, researchers and doctors have always tried to explain ways to extract blood from the body and separate its elements. One of the common methods for separating platelet elements from the blood is using the syringe to enter the patient's vessels, to create negative pressure and its subsequent extraction of blood, and after extracting the blood, the material was poured into the test tube using a centrifuge process and the difference in the particle mass and the constituent elements of the blood make to create different several layers and to become multi-stages of the blood possible. Now, by using a separating process, it's possible to extract the PRP and its induction. In another method, standard blood bags are used to separate the elements and extract the blood components. It will be followed by the represented inventions in the international field.

### DETAILED DESCRIPTION

According to the FIG. (1), by using the needle (100), Due to the intense pressure in the vein, blood flows through the needle path, blood flowed through the needle path and after passing the needle connection to the PRP kit tube and arrives to the internal lock (102) and it must be mentioned that in the drawing blood time, by breaking the mentioned lock, It's possible to enter the blood to bag blood, due to the openness of the external lock (103), blood can enter to the mentioned blood through the connected tube to the bag blood (105) and by passing the required time, the volume of blood assembled in the mentioned bag is accumulated. By completing an extraction volume of blood, the external lock (103) can be locked and by cutting the needle part (100) and internal lock (102) of the PRP set kit can be blended and put together such that, the output of the first blood bag (105) is in the face of the rotating central of centrifuge machine. By ending the centrifuge process, blood is divided into three parts. There are red blood cells and heavier elements at the below and which in this part forms the greater volume of blood. In the middle, after ending the centrifuge, there is relatively the less volume and thinner layer of blood that is included PRP section and the its below, there is a very thin layer of white blood cells and in the above, there are plasma and liquids like water.

Now, by breaking the second internal lock (109) and squeezing shuttle the form blood bag, the separated phases enter to the outlet duct of the first bag (108) and after passing the Three Way (109) the internal lock (110) enter into the second bag after passing the connected tube of the second bag (113) due to the openness of external Lock (112). According to shuttle form of the first bag, the height of each phase is multiplied when passing through the bag outlet and layer width is becoming very low as well as. This caused that operator has maximum precision and power to send off self-requested phases. After passing plasma phase, and also the transferring separated platelet phase from first Bag to second Bag, by locking the external lock that is located between two bags (112), the possibility of returning the separated phases and mixing the phases is eliminated.

Now by blending the PRP kit in the presented invention and second bag re-centrifuge, the same way as first bag, the possibility of separation of plasma and platelets is possible. Since there is a total amount of current kit space available for standard anticoagulants, there is no risk of blood coagulation or separated cells. Now by unlocking external lock between the two bags and squeezing the second bag, it can be possible to return plasma phase (PPP) to the first bag and after locking external lock between two bags in the first bag, we have common blood elements. If needed it can return to the patient's body by unlocking the connection (111) and by injecting. In the other hand, remaining PRP in the second bag (113) extracts by unlocking connection (116) and if needed it can be injected to the required position. The rigid bars (114) are located two sides of the bag prevent to squeeze the bag during the centrifuge process. The flexibility of the used polymer in making the present kit prevents the destruction of platelet shells.

### Description an Executive Sample

The presented invention is included at least at least one drawing blood needle, the collection of the locks before and after the blood bag, one shuttle form blood bag, one three way, two connections for extracting the material, one shuttle form blood bag that is equipped with side bars for the vertical and strong stand which because of the absence of negative pressure in the drawing blood process, platelet dissolves and reduces the effectiveness of PPP will be a minimum and also because of the spindle shape in the case of the first bag, there is a better control of the process of separating blood phases. Due to the presence of anticoagulant, the possibility of blood coagulation and its elements during the process are minimized and due to the presence of a connection of blood extraction from the first bag, if necessary, blood elements can be injected into the patient's body again and the Shuttle form of the second bag can provide high precision in the process of separating the extraction phases and returning these phases to the first bag and wherein the presence of rigid bars on the sides of the shuttle bag prevents the bag from being squeezed when centrifuging the second bag and the flexibility and softness of the polymer used in the production of bags minimizes the damage to the platelets.

1. A platelet-rich plasma (PRP) production kit for re-injecting remaining elements after separating PRP from blood, comprising:
a first blood bag;
at least one drawing blood needle;
a plurality of locks positioned before and after the first blood bag;
a three way coupler;
two connections for extracting material from the first blood bag; and
a second blood bag having a shuttle form that is equipped with side bars for standing the second blood bag in a vertical orientation.

2. The platelet-rich plasma (PRP) production kit of claim 1, wherein the production kit provides an absence of negative pressure in a blood drawing process such that the producing kit prevents dissolving of a platelet a reduction in an effectiveness of platelet-poor plasma (PPP).

3. The platelet-rich plasma (PRP) production kit of claim 1, wherein the first blood bag comprises a spindle shaped case that provides control during a separating blood of phases.

4. The platelet-rich plasma (PRP) production kit according to claim 1, further comprising an anticoagulant that minimizes blood coagulation.

5. The platelet-rich plasma (PRP) production kit according to claim 1, further comprising a connection for extracting blood from the first blood bag wherein the connection enables a re-injection of blood elements into a patient's body.

6. The platelet-rich plasma (PRP) production kit according to claim 1, wherein the second blood bag having a shuttle form provides precision during a separating of the extraction phases and returning the extraction phases to the first blood bag.

7. The platelet-rich plasma (PRP) production kit according to claim 1, further comprising rigid bars on sides of the second blood bag that prevent the second blood bag from being squeezed during a centrifuging of the second blood bag.

8. The platelet-rich plasma (PRP) production kit according to claim 1, wherein the first blood bag and the second blood bag are formed of a flexible and soft polymer that minimizes damage to platelets stored therein.

9. A method, comprising:
providing a platelet-rich plasma (PRP) production kit for re-injecting remaining elements after separating PRP from blood, comprising:
a first blood bag;
at least one drawing blood needle;
a plurality of locks positioned before and after the first blood bag;
a three way coupler;
two connections for extracting material from the first blood bag; and
a second blood bag having a shuttle form that is equipped with side bars for standing the second blood bag in a vertical orientation.
